(19) United States
(12) Patent Application Publication
(10) Pub. No.: US 2026/0024622 A1
(43) Pub. Date: Jan. 22, 2026

(54) METHOD AND APPARATUS

(71) Applicant: APOHA LIMITED, LONDON (GB)

(72) Inventors: SHAMIT SHRIVASTAVA, LONDON (GB); ALEC THOMAS, LONDON (GB); ALIREZA MEGHDADI, LONDON (GB)

(73) Assignee: APHOA LIMITED, LONDON (GB)

(21) Appl. No.: 19/339,909

(22) Filed: Sep. 25, 2025

Related U.S. Application Data
(63) Continuation of application No. PCT/GB2024/050857, filed on Mar. 28, 2024.

(30) Foreign Application Priority Data
Apr. 5, 2023 (GB) 2305103.0

(51) Int. Cl. G16B 40/10; G16B 15/30

(52) U.S. Cl. CPC G16B 40/10; G16B 15/30

(57) ABSTRACT

An aspect of the disclosure provides a method of biophysical characterisation of a biomolecule of a certain biomolecule class, or a composition thereof, said method comprising the steps of: i) obtaining wave data for a sample based on physical measurements of a wave generated, in a liquid system, by providing contact between a droplet of the sample and the liquid system, wherein the wave comprises modes encoding characteristics of the interaction between the sample and the liquid system, wherein the sample contains the biomolecule, or a composition thereof, for analysis; ii) providing a biophysical characterisation of the biomolecule or the composition comprising the biomolecule based on the wave data.

### FIELD OF INVENTION

The present invention relates to methods and apparatus, and more particularly to methods and apparatus for biophysical characterisation of biomolecules and compositions comprising such molecules.

### BACKGROUND

Biomolecules, such as proteins, fats, oils, and other biomolecules have a range of properties which make them useful. In relation to foodstuffs, a variety of compositions comprising biomolecules may be used to achieve certain desired properties. Proteins and fats for use in the food industry may have significant value.

In relation to personal care products and cosmetics biomolecules may be used in a variety of products such as rinse-off products including shampoos, soaps, toothpastes, and shower gels. They may also be used in “leave-on” products such as sanitizers, sunscreen lotion, cosmetics, body and face creams, insect repellent, perfumes, and antiperspirants.

Biomolecules may also be used in a variety of therapeutic compositions, for the therapeutic treatment of humans and animals.

The properties of biomolecules and the manner in which they will perform in certain circumstances of use may be difficult to determine. A major area of research is so-called “developability assessment”, which relates to the evaluation of a potential therapeutic candidates properties. Often the objective of such evaluation is to avoid late-stage failures in clinical research and to improve the manufacturability and safety profile of drugs.

The surface of a material has a thermodynamic potential that is independent of its volume. The physical and chemical properties of a surface are derived from its thermodynamic potential. For example, the response of the surface to a mechanical perturbation is given by properties such as surface tension and lateral compressibility. Similarly, the response of the surface to an electromagnetic perturbation is given by properties such as surface dipole moment. As a result of these perturbation, different types surface waves may be generated on a surface e.g. a surface of a fluid (e.g. a liquid) forming an interface with another fluid (e.g. air). Some example types of surface waves are: Rayleigh waves; Gravity waves; Capillary waves; Lucassen waves. The physics of these waves have been described in Nonlinear fractional waves at elastic interfaces Julian Kappler, Shamit Shrivastava, Matthias F. Schneider, and Roland R. Netz Phys. Rev. Fluids 2, 114804—Published 20 Nov. 2017. These waves may be hydrodynamically coupled.

Rayleigh waves are characterised by elliptical motion of a notional fluid particle in a plane which is perpendicular to the surface at equilibrium and parallel to the direction of propagation of the wave.

Gravity waves are characterised by a displacement from equilibrium of a notional fluid particle at the surface wherein the displacement of the notional particle is characterised by having a restoring force of gravity or buoyancy.

Capillary waves are characterised by a displacement from equilibrium of a notional fluid particle wherein the displacement of the notional fluid particle is in a direction transverse to the surface at equilibrium and transverse to the direction of propagation of the wave and have a restoring force of surface tension.

Lucassen waves are characterised by a displacement from equilibrium of a notional fluid particle at a surface of a wave-medium by oscillation in a direction parallel to that surface at equilibrium and parallel to the direction of propagation of the wave. In Lucassen waves this notional particle is subject to a restoring force resulting from the surface elastic modulus of the surface of the wave-medium. Put another way Lucassen waves are compression-rarefaction waves which occur in the plane of a boundary (an interface) between a wave-medium and an adjacent medium such as air.

Lucassen waves have been observed in lipid monolayers and in other types of liquid systems.

Shamit Shrivastava, Matthias F. Schneider Opto-Mechanical Coupling in Interfaces under Static and Propagative Conditions and Its Biological Implications describes how a wave can be generated in a lipid monolayer mechanically with a dipper and how parameters of the generated wave, such as the intensity of fluorescent particles therein and the lateral pressure of the surface wave, can be measured, for example using a photo detector and a Wilhemly balance respectively.

Shrivastava S, Schneider MF. 2014 Evidence for two-dimensional solitary sound waves in a lipid controlled interface and its implications for biological signalling. J. R. Soc. Interface 11:20140098 describes a method in which Lucassen waves can be generated in a lipid monolayer and how parameters of said waves may be measured (e.g. fluorescence energy transfer (FRET) measurements; a piezo cantilever). The document also describes how the state of a lipid monolayer may be characterised by a variety of thin film parameters (e.g. surface density of lipid molecules, temperature, pH, lipid-type, ion or protein adsorption, solvent incorporation, etc.) and also how the state of the lipid monolayer can affect parameters of waves which propagate in the lipid monolayer.

Bernhard Fichtl, Shamit Shrivastava & Matthias F. Schneider, Protons at the speed of sound: Predicting specific biological signaling from physics Nature Scientific Reports describes how Lucassen waves can be generated in a lipid interface in response to a change in pH of the system and that the speed of these waves can be controlled by the compressibility of the interface. The document describes how parameters of these waves depend on the degree of change in pH. The document also describes how mechanical and electrical changes at the lipid interface can be measured (e.g. using a Kelvin probe).

Lucassen waves may be described as interfacial compression waves and may be considered two-dimensional sound waves (sound waves confined to a surface which forms a boundary between two phases e.g. a fluid-air boundary). In a manner analogous to sound waves, shock waves may exist in Lucassen wave systems (e.g. two-dimensional shock waves). Lucassen shock waves may be characterised in the same way as Lucassen waves with the additional constraint that the waves are characterised by changes in the wave medium which are nonlinear and/or discontinuous.

S. Shrivastava, Shock and detonation waves at an interface and the collision of action potentials, Progress in Biophysics and Molecular Biology, describes how Lucassen shock waves may propagate through a lipid interface.

WO2019234437A1 describes how a lipid interface may be used to transmit and receive signals. The document describes a signal processing device comprising: a first medium; a second medium; a lipid interface arranged between the first medium and the second medium, wherein the lipid interface comprises a plurality of lipid molecules; an input transducer arranged to apply an input signal to the lipid interface, wherein the input signal is arranged to generate a mechanical pulse in the lipid interface; and an output transducer arranged to receive an output signal by detecting a mechanical response in the lipid interface from the mechanical pulse generated in the lipid interface by the input transducer; wherein the lipid interface is arranged to propagate the mechanical pulse from the input transducer via the lipid interface to the output transducer.

### SUMMARY

Aspects of the invention are set out in the independent claims and optional features arc set out in the dependent claims. Aspects of the disclosure may be provided in conjunction with each other and features of one aspect may be applied to other aspects.

### BRIEF DESCRIPTION OF DRAWINGS

Embodiments of the disclosure will now be described in detail with reference to the accompanying drawings, in which:

FIG. 1 shows a flow chart indicating the steps of a method for biophysical characterisation of a biomolecule or a composition including such a biomolecule;

FIG. 2 shows a diagram of an apparatus which may be used for performing the measurements of the physical wave in the liquid as described in embodiments of the present disclosure and comprising the hardware components employed in methods such as those described with reference to FIG. 1 and/or FIG. 2;

FIG. 3 shows a flow chart indicating the steps of a method of identifying a candidate biomolecule within a class of biomolecules, or a composition thereof, with comparable biophysical properties to a target biomolecule of the same class of biomolecule or a composition thereof;

FIG. 4 shows a plot of displacement of the liquid at a location (Y-axis) against time (X-axis) for two different samples. The first sample being of one glyceride oil, the second sample being the same glyceride oil adulterated with 0.1% of a different glyceride oil;

FIG. 5 shows a visual representation of an array of wave data in which the rows of the array each correspond to a waveform obtained by measuring a physical wave at a location in the liquid as a function of time; and

FIG. 6 shows a simplified visual representation of the identification of separable contributions of the variance in a data array such as that illustrated in FIG. 5;

FIG. 7 shows the results of principle component analysis (PCA) from wave data obtained for a selection of glyceride oils.

In the drawings like reference numerals are used to indicate like elements.

### SPECIFIC DESCRIPTION

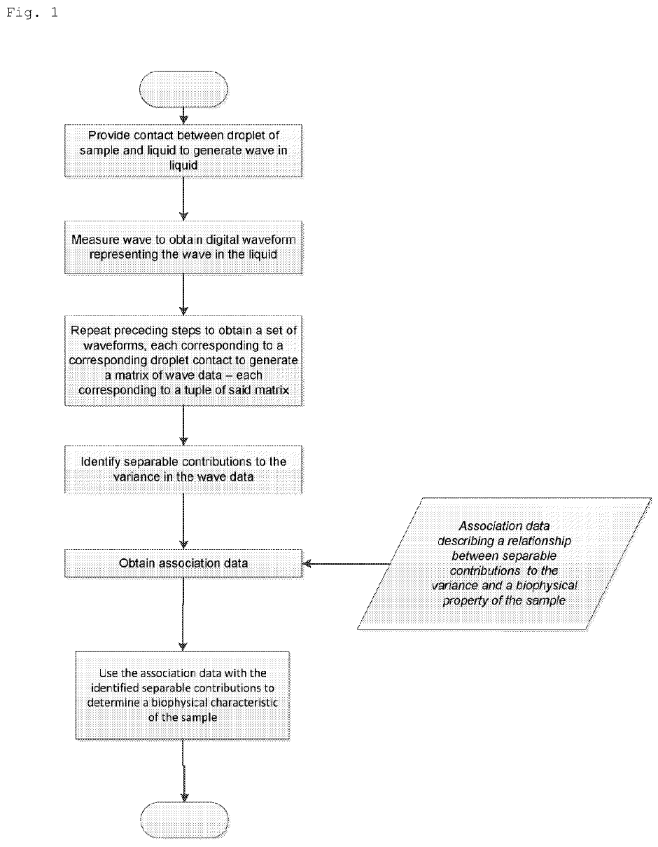

FIG. 1 illustrates a method of biophysical characterisation of a biomolecule of a certain biomolecule class, or a composition thereof. The characterisation is based on wave data, obtained from physical measurement of a wave (or waves) in a liquid generated by contact between the liquid and a droplet of that biomolecule or composition.

Initially, prior to beginning the method illustrated in FIG. 1, a sample is prepared comprising the biomolecule or composition, as described in more detail below.

A droplet of the prepared sample is then brought into contact with the liquid. This may comprise dropping the droplet from a dispenser so that it drops onto and impacts the liquid or the droplet may be lowered into a position in which it contacts the liquid, such as by moving a nozzle of the dispenser carrying the droplet. The droplet may also be generated in a position such that contact with the liquid occurs when the droplet reaches a given size. It will thus be appreciated that contact may take place at negligible net linear speed.

The liquid may be provided in a trough, such as a Langmuir trough or any similar system. The characteristics of the liquid will be described in detail below, but typically it comprises a liquid solution (such as an aqueous solution) and/or liquid suspension. Suspensions including colloidal suspensions may be used. A thin film may be provided at the surface of the liquid.

Contact between the droplet and the liquid generates a physical wave in the liquid. This wave is measured at a particular location, which typically is displaced a short distance from the location at which contact takes place. These measurements provide a time series of samples which describe disturbance of the liquid at that particular location. Typically, the measurements are performed optically or electrically but, however it is achieved, the measurement technique generally provides measurement of the physical wave which includes sensitivity to at least two or more of the following wave modes: Rayleigh waves, gravity waves, capillary waves and Lucassen waves. Typically, a sensitivity to Lucassen waves is desirable. These wave modes encode characteristics of the interaction between the sample and the liquid system which themselves depend on the biophysical properties of the biomolecule or composition in the sample. The wave data is stored in a data store, such as a digital memory, for retrieval by a processor. Typically, the wave data associated with the droplet comprises a waveform or set of waveforms which provide a measurement of the physical wave in the liquid having sufficient degrees of freedom to encode the relevant wave modes which make up that physical wave. A visual representation of two such waveforms is illustrated in FIG. 4.

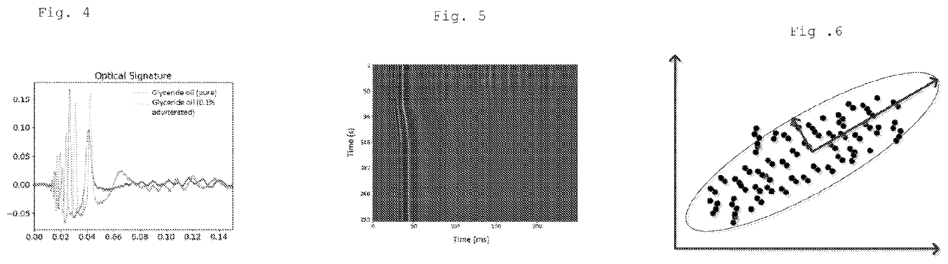

The above procedure may be repeated for a plurality of droplets, so for each droplet, that waveform or set of waveforms is stored into the data store. The condition of the liquid for each droplet may be identical to all preceding droplets. For example, the composition of the liquid at the location where contact occurs may be controlled so that it remains substantially equal for all droplets. One way to achieve this is to provide flow in the liquid, but other methods may be used. It is also possible however to allow the biomolecule or composition to accumulate in the liquid, so that its concentration at the location where contact occurs increases as droplets are applied to the liquid. In any event, the end result of this procedure is a set of tuples of wave data. Each tuple corresponding to the waveform(s) obtained by measuring the physical wave caused in the liquid by a corresponding one of the droplets. This may be stored as an array of data in which the rows or columns correspond to such tuples. A visual representation of such an array is provided in FIG. 5.

This array of wave data is then operated on by a data processor, which may be configured to identify separable contributions to the waveforms described in that data. This may be done by a dimensionality reduction technique, for example, the processor may be configured to apply a principal components analysis (PCA) to the wave data. It will be appreciated in the context of the present disclosure that such analysis generates a number of coefficients and a number of vectors. For example, the coefficients, p, may be the eigenvalues and the vectors, P, the eigenvectors of the covariance matrix of the wave data. Other dimensionality reduction techniques may be used to identify one or more separable contributions to the variance in the wave data. A visual representation of dimensionality reduction of wave data is provided in FIG. 6.

Next, association data is obtained from a data store, such as digital memory. The association data may describe a predetermined relationship between characteristics of the wave data and one or more biophysical characteristics of the sample. For example, it may provide a mapping from the separable contributions to particular values of the biophysical characteristics.

The method may comprise the data processor obtaining, e.g. from input, information defining the class of biomolecule and selecting the relevant model from the association data according to the class of biomolecule. The association data may store a set of models for any one or more of the following classes of biomolecules:
polymeric and/or macromolecular classes of biomolecules, such as polypeptides, lipids, polysaccharides, and nucleic acids.
proteins, such as:
   antibodies, contractile proteins, enzymes, hormonal proteins, structural proteins, storage proteins, and transport proteins; or
   plant protein, which may be selected from algae protein, black bean protein, canola wheat protein, chickpea protein, fava protein, lentil protein, lupin bean protein, mung bean protein, oat protein, pea protein, potato protein, rice protein, soy protein, sunflower seed protein, wheat protein, white bean protein, and protein isolates or concentrates thereof
   animal protein, which may be selected from porcine protein, bovine protein, ovine protein, equine protein, avian protein and protein isolates or concentrates thereof
non-polymeric and/or non-macromolecular classes of biomolecules, such as biomolecules selected from vitamins, phytochemicals (e.g. carotenoids, flavonoids, isoflavonoids, and phenolic acids), food flavourings (e.g. those based on plant-derived alcohol, ester and/or ketone functional compounds), amino acids, peptides, mono-, di- and oligo-saccharides, free fatty acids, mono-, di-, and triglycerides; sterols, steroids, nucleotides and nucleosides, such as:
   edible glyceride oil selected from vegetable oils, marine oils, and animal oils such as a vegetable oil selected from açai oil, almond oil, beech oil, cashew oil, coconut oil, colza oil, corn oil, cottonseed oil, grapefruit seed oil, grape seed oil, hazelnut oil, hemp oil, lemon oil, macadamia oil, mustard oil, olive oil, orange oil, peanut oil, pecan oil, pine nut oil, pistachio oil, poppyseed oil, rapeseed oil, rice bran oil, safflower oil, sesame oil, soybean oil, sunflower oil, walnut oil and wheat germ oil.

It will be appreciated in the context of the present disclosure that these are merely examples of the classes of biomolecules to which the present disclosure may be applied.

The data processor may also be configured to select within the set of models stored in the association data according to the biophysical parameter (or parameters) which is/are to be determined for that class.

Next, the association data for the relevant class of biomolecule or composition and the relevant biophysical characteristic(s) is used, with the wave data, to determine data indicating those biophysical characteristic(s). This may comprise performing operations, defined in the association data, on data indicating the separable contributions.

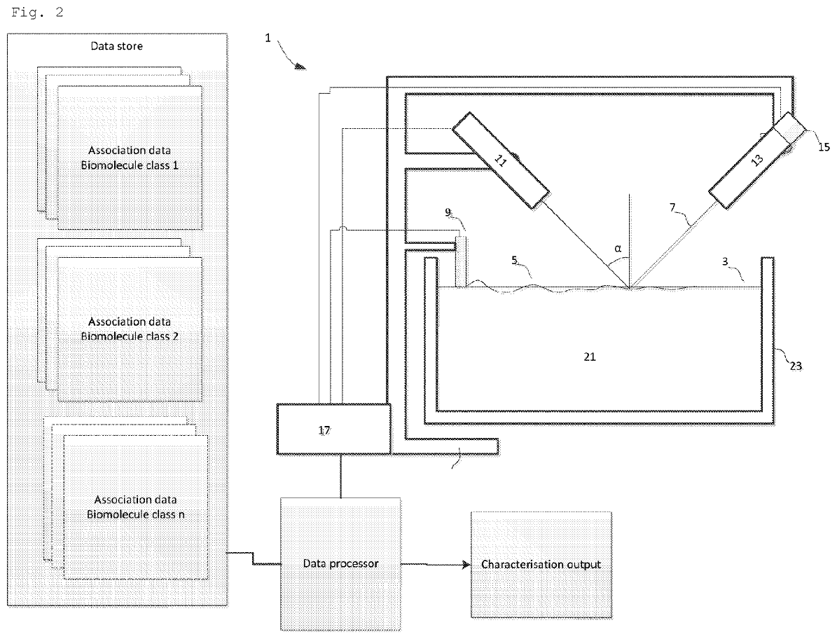

An apparatus to implement this method is depicted in FIG. 2. In this example, the source of the wave data in the apparatus illustrated in FIG. 2 is an analytical instrument 1 configured and arranged to perform physical measurements of the wave in the liquid which is generated by contact between the liquid and a droplet of the sample.

As illustrated, the instrument 1 is configured to generate the wave data as described below and to provide that wave data to the data processor.

The data processor comprises an input connection for obtaining the wave data from a data source such as the instrument 1. The data processor is also connected to a data store, such as a digital memory, storing the association data.

The association data may be based on prior analyses of other biomolecules of the same class of biomolecules as are present in the sample. As illustrated, the association data may comprise a plurality of sets of models—each set of models relating to a certain class of biomolecules and compositions thereof. Each such model comprises digital data defining data operations to be performed by the processor to provide a mapping between the wave data and one or more biophysical characteristics.

A variety of such models may be used. As a first example, the association data may comprise a plurality of coefficients defining a linear model of one or more biophysical characteristics as a function of selected separable characteristics of the wave data. The data processor may be configured to determine the biophysical characteristic by scaling each of the selected contributions, p, according to a corresponding one of the coefficients in the association data and then making a linear sum of the scaled contributions. As a second example, the model may be non-linear and may define the one or more biophysical characteristics as a non-linear function of the selected contributions, p, and a data processor may apply the non-linear function to those separable contributions, p, to determine the biophysical characteristic. As a third example, the association data may define an adaptive model, for example it may comprise a machine learning element in the form of a network of nodes and/or weights and the data processor may be configured to apply that machine learning element to the wave data to determine the biophysical characteristic of the biomolecule or composition in the sample. The nodes in that network may each comprise the linear and/or non-linear operations as described above and may also comprise dimensionality reduction processes to identify separable components for use in such operations. For example, the association data and the data processor together may be configured to implement a machine learning element, such as a neural network or other machine learning system.

The association data may comprise a set of such models for each class of biomolecule and for compositions comprising biomolecules of that class, for example the association data may comprise a set of models for proteins, a set for lipids, and so on. Each model in each set may relate one or more predetermined biophysical characteristics of biomolecules or compositions of that class to characteristics of the wave data for biomolecules of that class, such as selected separable components of such wave data.

The data processor is configured to obtain, e.g. from input or hardcoded data, information defining the class of biomolecule. It is further configured to select the relevant model from the association data according to the class of biomolecule. The data processor may also be configured to select within the set according to the biophysical parameter (or parameters) which is/are to be determined for that class.

As a first example, where the class of biomolecule is a food protein, the data processor may be configured to select the association data related to protein classification and to select, within that association data, the model(s) associated with one or more sensory properties exhibited by the biomolecule or a composition comprising the biomolecule in use in a food product.

As a second example, where the class of biomolecule or composition thereof is a protein which is a therapeutic candidate, such as an antibody or hormonal protein, the data processor may be configured to select the association data related to protein classification and to select within that association data, the model(s) associated with
a metric of developability, such as manufacturability or safety profile.

As a third example, the biomolecule may be an edible glyceride oil selected from vegetable oils, marine oils, and animal oils the data processor may be configured to select the association data related to a sensory property associated with application of the biomolecule, or a composition thereof, to the human or animal body. Examples of such sensory properties include: skin absorption, skin spread, skin stickiness, skin thickness, in use limpness, in use shine, in use smoothness, in use softness, post wash frizz, post wash shine post wash smoothness, and post wash softness.

As a fourth example, for any class of biomolecules or composition thereof the data processor may be configured to select the association data related to quantitative properties selected from viscosity, stability in a particular environment, hydrophobicity, binding affinity to a particular target, solubility in a particular solvent, surface charge, and specific gravity.

The data processor is further configured to perform data operations defined by the selected association data for the relevant class of biomolecule or composition on the wave data to determine data indicating the biophysical characteristic.

As described above, dimensionality techniques can be used to identify separable contributions of the wave data and then the separable contributions can be used to draw inferences about the biophysical characteristics of the biomolecule or composition. Examples of dimensionality reduction method comprises a blind signal separation, BSS, methods such as: principal component analysis; singular value decomposition; independent component analysis; and non-negative matrix factorization. Other examples of dimensionality reduction methods comprise spectral analysis and multi-resolution analysis methods, such as time-frequency analysis (including analysis based on a short-time fourier transform and wavelet analysis). However, it is not necessary to use a separate dimensionality reduction step or indeed to perform dimensionality reduction at all. A machine learning element, such as a neural network or other machine learning system, may comprise an embedding to reduce its dimensionality as part of its integrated data processing functions rather than as a separate preceding step. In some embodiments a machine learning element may be trained to attribute one or more biophysical characteristics of biomolecules of a certain class, or compositions thereof, to wave data. In these embodiments, there is no need to determine the separable contributions by dimensionality reduction or otherwise. The association data may define a relation between the wave data and the biophysical characteristic. Machine learning is just one way to do this—the association data may define other mappings between wave data and biophysical characteristics so as to act on the wave data to provide a biophysical characterisation of the biomolecule or the composition comprising the biomolecule.

It will be appreciated in the context of the foregoing disclosure that the data processor may be configured to act on data obtained directly from an instrument 1 such as that illustrated in FIG. 2. For example, the methods of the present disclosure may comprise performing the physical measurements of the wave, such as by preparing the sample for analysis, providing the contact between the droplet of the sample and the liquid system to generate the wave, and then also performing the physical measurements of the wave. However, the physical measurement may have been performed separately by another instrument, and the data processor may be configured to act on data obtained from other sources such as retrieval from memory or received via a network message.

The physical wave may comprise a surface wave comprising modes encoding characteristics of the interaction between the sample and the liquid. Examples of such wave modes include a Lucassen wave mode. Whatever instrument or measurement method is used, the physical measurement may comprise a sensitivity to such modes, optionally including a Lucassen wave mode.

The instrument 1 is one example of an apparatus for characterising an interaction between a droplet of a sample and a liquid system. The apparatus characterises a wave 5 in the liquid system based on physical measurement of the wave. This may be done by optical methods such as by measuring the polarisation of a light beam 7 reflected by the wave in particular it may use a ratio between the s-polarisation component and the p-polarisation component of the reflected light beam 7 to sense variations in refractive index (and hence density) of the liquid in the presence of the wave. A time series of such data at a particular location in the liquid can be used to characterise waves.

The apparatus 1 shown in FIG. 2 comprises, a droplet provider 9 configured to contact the liquid with a droplet of a sample, light beam optics 11, a light collector 13, a detector 15, and a wave measurement module 17. As illustrated in FIG. 2, the apparatus 1 also comprises a reservoir 23 holding a volume of liquid 21 (i.e. the reservoir medium). The reservoir 23 may be provided by a trough, such as a Langmuir trough.

Also shown in FIG. 2 mechanical fixtures 19 may be provided for holding the apparatus in position with respect to the reservoir 23, but it will be appreciated that these fixtures 19 are not essential and may be made and sold separately from the apparatus 1 itself. The wave measurement module 17 is connected to the droplet provider 9 and to the detector 15 for the communication of control signals and data, it may also be connected to the light beam optics 11.

Examples of types of liquid which may provide the volume of liquid 21 (i.e. the reservoir medium) comprise aqueous solutions (e.g. deionized and purified water) and suspensions.

The light beam optics 11 comprise a source of polarised light arranged to illuminate an area of the liquid surface 3 with a beam 7 having a selected angle of incidence a. The light beam 7 may also be coherent. Examples of suitable light sources include lasers and the light beam optics may comprise a polariser.

The light collector 13 is arranged to receive the beam of light 7 after reflection by the area of the thin film and to provide the reflected beam of light to the detector. The light collector 13 is positioned so that the optical axis of the light collector 13 is directed to the area of the liquid surface 3 at the angle of specular reflection, a, of the light beam incident on the liquid surface from the light beam optics.

The detector 15 is configured to sense parameters of the light received from the light collector 13 and to provide signals to the wave measurement module 17 including those parameters. Typically, those parameters comprise parameters capable of characterising the wave and may comprise a sensitivity to a variety of wave modes such as Lucassen waves and other types of wave. For example, the parameters may indicate the polarisation of the light beam 7 after interaction with the wave. For example, the parameters may comprise a measure of the intensity of one or more polarisation components of the received light, such as the intensity of (a) an first component of the second polarisation and/or (b) a second component of the second polarisation. The second component may be orthogonal to the first component. The first component may be the s-component and the second component may be the p-component.

The droplet provider 9 is positioned with respect to the liquid 5 so that it can provide contact between the liquid and a droplet of the sample. For example, the droplet provider 9 may comprise a source of the sample and may be configured to contact the surface of the liquid with a droplet of the sample to generate a wave in the liquid. Such stimulus may create a wave 5 in the liquid exhibiting some or all of the above wave modes.

The wave measurement module 17 is configured to control the droplet provider 9 to apply the stimulus to the liquid, and to operate the detector 15 to collect a time series of samples of the light received at the detector 15. These samples may comprise samples of the intensity of the one or more polarisation components mentioned above. Typically the sample rate is at least 1 MHz, for example 10 MHz. The wave measurement module may also be configured to apply a low pass filter to the time series before down-sampling the data to 20 kHz or thereabouts. Typically, the sample rate of the down-sampled time series is selected based on the size of the illuminated area and the expected speed of the wave in the liquid surface. For example, the expected speed may be approximately 1 ms-1 and the illuminated area may have a diameter of ˜5 mm, in which case the upper limit on the frequency of surface waves that can be meaningfully sampled will be 10 kHz. The sample rate of the down-sampled time series may be selected to ensure that the measurement remains well within this available bandwidth.

The wave measurement module 17 may be configured to control the timing of these samples based on the operation of the droplet provider 9, for example so that the surface wave 5 in the area of the liquid surface illuminated by the beam 7 can be sampled at a selected time after the contact with the droplet and for a selected duration. The time and/or duration typically are selected based on the distance from the part of the liquid to which the droplet is applied to the illuminated area. The wave measurement module may be further configured to provide a particular sampling scheme for a particular measurement type. The wave measurement module may be configured to implement a first sampling scheme to perform a first measurement type and to implement a second, different, sampling scheme to perform a second, different, measurement type. For example, where a thin film is present at the surface of the reservoir, to measure viscosity or hydrophobicity in a lipid thin film, or to measure binding in a protein thin film, the wave measurement module may use a long sampling duration (total time for which samples are collected). Recording of a single stimulus typically has a time resolution of microsecond and duration of seconds. This can be sufficient for measurement of properties of molecule that interact strongly with the film and/or are fast, for example electrostatic interaction or hydrogen bonding. In some embodiments multiple such stimulations with a repetition rate of few seconds observed over a course of minutes to hours could provide improved measurement of properties of molecules that interact weakly and/or slowly with the film, for example binding or reaction kinetics.

The wave measurement module may be configured to sample data in a selected time interval following the application of a stimulus to the liquid surface of the reservoir, and to repeat the same sampling in that same time interval after subsequent stimuli to provide repeated measurements. Such measurements may be of relatively short duration.

In operation, the wave measurement module 17 operates the droplet provider 9 to provide contact between the liquid and a droplet of the sample. This triggers a wave 5 in the liquid. The wave travels outwardly, across the liquid from the location at which the contact takes place. The light beam optics 11 illuminate an area of the liquid through which the wave travels, and the wave measurement module 17 operates the detector 15 to take a series of samples which represent the light beam 7 after its interaction with the area. These may be provided by the light collector 13 to the detector 15. Accordingly, the disturbance of the liquid by the wave at the location can be recorded as a function of time in a series of samples of data (a time series). Each sample in that time series may comprise polarisation data, which may be in the form of the intensity of the s-polarisation component and the intensity of the p-polarisation component of the reflected light. The wave measurement module 17 may be configured to determine an indication of the polarisation angle of the reflected beam 7, such as a ratio of the intensity of the s-component to the intensity of the p-component for each sample. The wave measurement module may derive features of the Lucassen wave from this time series. Examples of features of the Lucassen wave include its amplitude, frequency content, phase velocity, group velocity, phase and so forth. The wave measurement module 17 may then use these features of the Lucassen wave to provide information about the stimulus or about an optional thin film at the reservoir surface, as described below. Other methods of wave measurement may be used. This is just one example. Imaging methods and non-optical methods may be used including x-ray methods, electrical transducers and mechanical transducers.

The apparatus may be controlled to provide a series of such measurements of a series of such waves, each wave being generated by contact between the liquid and a droplet of the test substance. This may generate wave data comprising a plurality of individual waveforms, each corresponding to the physical measurement of one wave, generated in the liquid by contact with the droplet of sample.

It will be appreciated in the context of the present disclosure that the change in polarisation caused by reflection at an interface is related to the refractive index differences at the interface. The inventors in the present case have appreciated that refractive index may be related to the surface characteristics of a liquid. Accordingly, the wave measurement module can derive, from the time series of samples, such as the s-p ratio, information about variations in those characteristics as a function of time. This may enable complex wave modes with many degrees of freedom to be characterised. The reservoir surface may carry a thin film, and the characteristics of the interface which are measured to generate the wave data may be associated with variations in density and/or position of that thin film, as well as the orientation of molecules within the film. As will be appreciated, following deposition of the biomolecule, the biomolecule itself may populate a pre-existing thin film, or a thin film may be generated from the biomolecule itself.

Where a thin film is present typically, the thin film comprises a type of liquid which is different from that of the volume of liquid 21. The liquid thin film and the volume of liquid 21 may therefore have an interface between them such as a liquid-liquid interface. The thin film may have viscoelastic properties. These and other types of thin films may exhibit a variety of surface wave modes in response to stimulus. Examples of such wave modes comprise Rayleigh waves, gravity waves, capillary waves and Lucassen waves.

Examples of types of liquid which provide the thin film include proteins and lipids and other types of liquid. It will be appreciated in the context of the present disclosure that such materials may also be held (e.g., dispersed in suspension or otherwise) in the volume of liquid and dynamic equilibrium may exist between the thin film and the material held in the volume of liquid 21.

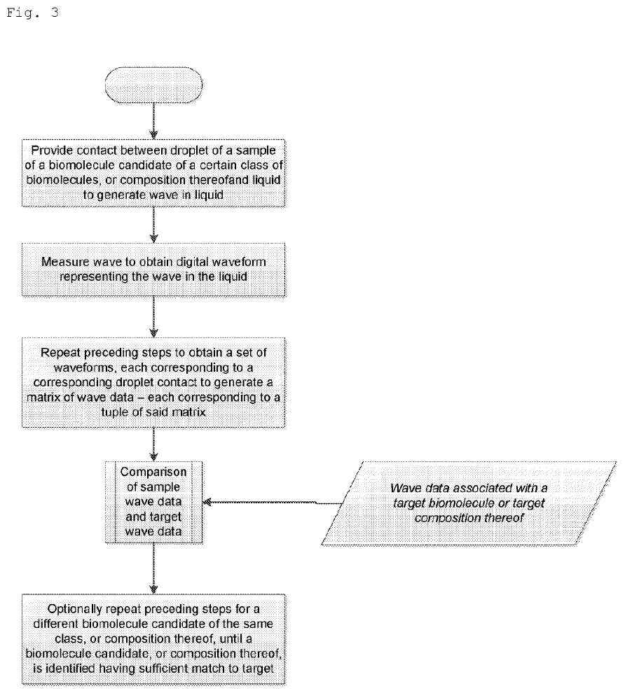

The above apparatus may be used in other methods. One such method is depicted in the flow chart illustrated in FIG. 3.

FIG. 3 illustrates a method of identifying a candidate biomolecule within a class of biomolecules, or a composition thereof, with comparable biophysical properties to a target biomolecule of the same class of biomolecule, or composition thereof.

As described above with reference to FIG. 1, a sample comprising a candidate biomolecule within a class of biomolecules, or a composition thereof may be prepared and contact may be provided between a droplet of such a sample and a liquid. The physical wave generated in the liquid by that contact may be measured to obtain physical wave data as also described above.

However, that wave data which is obtained comprises modes encoding characteristics of the interaction between the sample and the liquid system.

A data processor, such as that described above with reference to FIG. 2 then provides a comparison of the wave data obtained from interaction of the sample with the surface of the liquid system with wave data associated with the target biomolecule, or target composition thereof.

This comparison may be based on (a) a biophysical characterisation of the biomolecule or the composition comprising the biomolecule based on the wave data; and (b) a corresponding characterisation of the target biomolecule, or target composition thereof. Any of the biophysical characterisations described above may be used.

The above steps can then be repeated for each of a number of different biomolecule candidates of the same class, or compositions thereof, until a biomolecule candidate, or composition thereof, is identified having values of selected biophysical characteristics, indicative of properties of the candidate biomolecule, which match those of the target biomolecule.

The nature of this matching process may depend on the class of biomolecule in question. For example, developability of therapeutic candidates may be assessed in this way. Also, sensory properties of candidate biomolecules for use in food products or in personal care products may also be assessed in this way.

As described herein, in one aspect, there is provided a method of identifying a candidate biomolecule within a class of biomolecules, or a composition thereof, with comparable biophysical properties to a target biomolecule of the same class of biomolecule, or composition thereof. The method involves a comparison of the wave data obtained from interaction of a sample of the biomolecule, or composition thereof, with the surface of a liquid system with wave data associated with the target biomolecule, or target composition thereof. In another aspect, a method is provided in which a biophysical characterisation of a biomolecule is made based on wave data derived from the interaction of a biomolecule sample and a liquid system. The results of the biophysical characterisation may be incorporated as reference data within a library, against which the wave data of other biomolecules, or compositions thereof, may be compared.

In order to facilitate comparisons between wave data obtained for a target biomolecule, or reference biomolecule held in a library, the biomolecule sample which is tested is selected such that a liquid carrier present in the sample is substantially the same as that which is employed in acquiring the corresponding wave data of the target or reference biomolecule. Similarly, consistency in the liquid medium of the reservoir of the liquid system, as well as height of the droplet provider and volumetric flow rate thereof discussed herein, also facilitate a comparison. Nevertheless, this is not essential since comparisons may still be made with contributions from any variation in the method of wave data acquisition being accounted for computationally (e.g. by multidimensional calibration). Further, discussion of particular biomolecules useful in the methods described herein, together with discussion of sample preparation is provided below.

### Biomolecules

The methods described herein allow for biophysical characterisation of biomolecules, or compositions thereof, based on wave data that is obtained in connection therewith. Biomolecules are well-known organic molecules, primarily composed of hydrogen and carbon, which are found in nature, typically in organisms and/or form part of biological processes. There is no particular limit on the nature of the biomolecules that can be used in the methods described herein, or compositions comprising them.

In general, biomolecules may be assigned to polymeric and/or macromolecular classes, or alternatively non-polymeric and/or non-macromolecular classes. Polymeric and/or macromolecular classes of biomolecules include those selected from polypeptides, lipids, polysaccharides, and nucleic acids.

Proteins correspond to a certain form of polypeptides, specifically those having tertiary structure contributing to their function or use in a biological context. Examples of proteins include, for instance, antibodies, contractile proteins, enzymes, hormonal proteins, structural proteins, storage proteins, and transport proteins. As will be appreciated, the protein class encompasses a wide range of uses/functions, including therapeutic, cosmetic and nutritional applications.

One such form of protein biomolecule which has found substantial therapeutic value is of course antibodies (e.g. monoclonal or polyclonal antibodies). The methods of the present disclosure may be used to probe the biophysical characteristics of known antibodies, or those under clinical development. This can in turn represent a means for determining the viability of a candidate antibody under development from an earlier stage, based on a comparison of biophysical characteristics of the candidate versus a target antibody (e.g. a commercially available antibody with a proven therapeutic efficacy).

In some embodiments, the biomolecule (e.g. a protein biomolecule), or composition thereof, is a therapeutic candidate, such as an antibody or an hormonal protein. In some embodiments, the biophysical characteristic provided by the methods described herein comprises a metric of developability, such as manufacturability or safety profile. For example, the metric of developability is based on a plurality of biophysical characteristics, said plurality of biophysical characteristics each being derived from one or more separable contributions of the wave data.

The methods of the present disclosure may be used to assess the biophysical characteristics of animal proteins, which may be selected from porcine protein, bovine protein, ovine protein, equine protein, avian protein and protein isolates or concentrates thereof.

The increasing popularity and environmental benefits of a plant-based diet have, for instance, attracted interest from food manufacturers looking to mimic the sensory properties (e.g. organoleptic properties) of animal proteins with plant-based alternatives. The methods of the present disclosure may be used to assess the biophysical characteristics of a plant-protein, or composition thereof, for the purpose of comparison with a target biomolecule, such as an animal protein or alternative plant-protein, or composition thereof. Examples of plant proteins include those selected from algae protein, black bean protein, canola wheat protein, chickpea protein, fava protein, lentil protein, lupin bean protein, mung bean protein, oat protein, pea protein, potato protein, rice protein, soy protein, sunflower seed protein, wheat protein, white bean protein, and protein isolates or concentrates thereof.

Thus, in some embodiments, the methods described herein provide a biophysical characterisation of the biomolecule, or composition thereof, which comprises a sensory property of the biomolecule, or a composition thereof. For example, the biomolecule, or composition thereof, is selected from the protein class, and the method provides a biophysical characterisation which comprises a sensory property which indicates a property exhibited by the biomolecule or a composition comprising the biomolecule in use in a food product.

Examples of non-polymeric and/or non-macromolecular classes of biomolecule that may characterised using the methods of the present disclosure include those selected from vitamins, phytochemicals (e.g. carotenoids, flavonoids, isoflavonoids, and phenolic acids), food flavourings (e.g. those based on plant-derived alcohol, ester and/or ketone functional compounds), amino acids, peptides, mono-, di- and oligo-saccharides, free fatty acids, mono-, di-, and triglycerides; sterols, steroids, nucleotides and nucleosides.

A class of biomolecules that is of particular interest to the food and cosmetic industry includes glyceride oils that may be incorporated into food products or applied to the human or animal body. The term “glyceride oil” used herein refers to an oil or fat which comprises triglycerides as the major component thereof. For example, the triglyceride component may be at least 50 wt. % of the glyceride oil. The glyceride oil may also include mono- and/or di-glycerides. The glyceride oil is at least partially obtained from a natural source (for example, a plant, animal or fish/crustacean source). Glyceride oils include vegetable oils, marine oils and animal oils/fats which typically also include free fatty acid and phospholipid components in their crude form.

Vegetable oils include all plant, nut and seed oils. Examples of suitable vegetable oils which may be of use in the methods described herein include: açai oil, almond oil, beech oil, cashew oil, coconut oil, colza oil, corn oil, cottonseed oil, grapefruit seed oil, grape seed oil, hazelnut oil, hemp oil, lemon oil, macadamia oil, mustard oil, olive oil, orange oil, palm oil, peanut oil, pecan oil, pine nut oil, pistachio oil, poppyseed oil, rapeseed oil, rice bran oil, safflower oil, sesame oil, soybean oil, sunflower oil, walnut oil and wheat germ oil. Suitable marine oils include oils derived from the tissues of oily fish or crustaceans (e.g. krill). Examples of suitable animal oils/fats include pig fat (lard), duck fat, goose fat, tallow oil, and butter.

Free fatty acids which may be present in the glyceride oils include monounsaturated, polyunsaturated and saturated free fatty acids. Examples of unsaturated free fatty acids include: myristoleic acid, palmitoleic acid, sapienic acid, oleic acid, elaidic acid, vaccenic acid, linoleic acid, linoelaidic acid, α-linolenic acid, arachidonic acid, eicosapentaenoic acid, erucic acid and docosahexaenoic acid. Examples of saturated free fatty acids include: caprylic acid, capric acid, undecylic acid, lauric acid, tridecylic acid, myristic acid, palmitic acid, margaric acid, stearic acid, nonadecylic acid, arachidic acid, hencicosylic acid, behenic acid, lignoceric acid and cerotic acid.

Accessing a biophysical signature of a biomolecule, such as a glyceride oil, or complex composition thereof, through the methods of the present disclosure can enable a comparison with a target biomolecule of the same class (e.g. another glyceride oil) which has particular biophysical characteristics of interest, e.g. a desirable sensory profile.

Thus, in some embodiments, the biomolecule, or composition thereof, is an edible glyceride oil and the methods provide a biophysical characterisation of the biomolecule, or composition thereof, which comprises a sensory property of the biomolecule, or a composition thereof. For example, the sensory property may, for instance, indicate a property associated with application of the biomolecule, or a composition thereof, to the human or animal body. For example, the sensory property indicates least one of: skin absorption, skin spread, skin stickiness, skin thickness, in use limpness, in use shine, in use smoothness, in use softness, post wash frizz, post wash shine post wash smoothness, and post wash softness.

As will be appreciated, the methods described herein may also be used to assess any potential level of contamination across a series of biomolecule samples, and the extent of the contamination may be assessed based on the effect of the contamination on the prevailing biophysical characteristics.

### Biomolecule Sample

The methods of the present invention involve the generation of wave data in a liquid system-employing a liquid sample in which a biomolecule, or composition thereof, is incorporated into a liquid carrier. As will be appreciated, the particular nature of the biomolecule, or composition thereof, will impact the most suitable form the sample may take—i.e. the particular way in which the biomolecule is disposed within a liquid carrier. For example, the biomolecule may be incorporated in solution, as a suspension, or as part of an emulsion, depending on the miscibility of the biomolecule in a given liquid carrier, or the melting point of the biomolecule. The skilled person is readily able to select a liquid carrier based on the particular class of biomolecule being investigated.

For instance, for polar biomolecules, an aqueous liquid carrier is generally most appropriate, such as water (e.g. deionized water), a glycerol-water mixture, or saline (e.g. phosphate buffered saline (PBS)) or medical buffer solutions (e.g. tris (hydroxymethyl) aminomethane buffer (THAM)). Alternative non-aqueous liquid carriers, which may also be used solubilize amphiphilic and hydrophobic biomolecules include alcohols (e.g. methanol, ethanol and 2-propanol), chloroform, acetone and combinations thereof (e.g. methanol-chloroform combinations).

Biomolecules which are amphiphilic in nature (e.g. glycerides, free fatty acids, or lipids) may also suitably be incorporated into a sample as an emulsion (e.g. as an oil-in-water type emulsion), which may be generated by known methods. For instance, such an emulsion may be readily prepared by mixing the biomolecule, or composition thereof, with an aqueous solvent (e.g. deionized water), agitating the mixture (e.g. using sonication) and applying heat (e.g. from 30 to 60° C.) until an emulsion is formed, as indicated by a change to an opaque liquid.

Table 1 below sets out suitable liquid carriers for different classes of biomolecule, a suitable concentration range (w/v) of the biomolecule, or composition thereof, in the liquid carrier, as well as an indication of whether the sample would suitably form an emulsion.

TABLE 1

| | | Concen- | |
|---|---|---|---|
| | | tration | Sample |
| | | range | emul- |
| Biomolecule Class | Sample Liquid Carrier | (mg/mL) | sified? |
| Polypeptides (e.g. | water or PBS* | 0.1-1 | No |
| proteins, antibodies), | | | |
| peptides and/or amino | | | |
| acids | | | |
| Nucleic Acids | PBS* or THAM** | 0.1-1 | No |
| Lipids | PBS* | 0.1-5 | Yes |
| | Chloroform, chloroform- | 1-20 | No |
| | methanol combinations, | | |
| | or acetone | | |
| Poly- and/or oligo- | PBS* or water | 0.1-25 | No |
| saccharides | | | |
| Mono-, di-, and | water | 1-10 | Yes |
| triglycerides; and/ | | | |
| or free fatty acids | | | |
| Steroids | ethanol, 2-propanol, | 0.1-0.5 | No |
| | acetone | | |
| Lipids + Steroid mixture | PBS* | 0.1-5 | Yes |
| Nucleotides and/or | PBS* or THAM** | 0.1-1 | No |
| nucleosides | | | |

*= phosphate buffered saline
**= tris(hydroxymethyl) aminomethane buffer

### Reservoir Medium

As described briefly above, the reservoir medium employed in the methods described herein is a stable liquid (over the timescale of the experiment), typically a solution or suspension, through which surface waves can be propagated upon deposition of a sample therein. For ease of operation and reproducibility, simple aqueous solutions are typically employed in the method described herein. For example, deionized water or a brine/alkali metal halide salt solution (e.g. NaCl) may be used, the latter being of potential benefit in encouraging thin film retention/formation at the surface of the liquid reservoir, where present or desired. Surfactants may also be included in the liquid medium for the purpose of forming a thin film at the liquid reservoir surface as a means to modify or optimise the waveform response following sample deposition, if desired. Examples of suitable surfactants include anionic surfactants (e.g. those containing a sulfate or sulfonate groups).

### Biophysical Characterisation

The methods described herein are capable of providing biophysical characterisation of a biomolecule, or composition thereof, based on wave data generated in connection therewith. These characteristics include several different categories of characteristics, including those that directly affect the droplet formation and spread in the liquid system (i.e. relating to fluid dynamic parameters), as well as properties of compressible fluids, such as compressibility, bulk (second) viscosity, thermal conductivity, and heat capacity that play a role in wave propagation. In addition, there are enthalpies of interaction between the droplet and the liquid in the reservoir which directly contribute energy into the wave, and which are therefore derivable from the wave data, particularly enthalpies related to interfaces such as surface charge of the droplet, partition coefficient (hydrophobicity), and surface pKa. Kinetics and timescales of these interactions are also accessible (and are some of the above properties are time timescale dependent properties). Timescales may, for instance, carry information about the conformational changes in a system. Short timescales (i.e. <microsecond) may correspond to enthalpy of intramolecular conformation changes; medium timescales (i.e. microsecond to millisecond) may correspond to collective intramolecular changes in large molecules (e.g. allosteric changes); and long timescales (i.e. >millisecond) may be intramolecular as well as intermolecular collective changes (e.g. in a fluid to gel phase change).

The wave data is also capable of assessing indirect properties of the biomolecule, based on comparison to the wave data of a notional standard biomolecule, where incremental compositional changes away from the composition of the notational standard can give indirect insights into biophysical properties, such as freezing point depression, vapor pressure (volatility), osmotic pressure, boiling point elevation, critical micellization concentration, and surface pressure.

Thus, in another aspect, there is provided a method for determining enthalpies of interaction for a sample of a biomolecule of a certain biomolecule class, or a composition thereof, with a liquid system, said method comprising the steps of:
i) obtaining wave data for a sample based on physical measurements of a wave generated, in a liquid system, by providing contact between a droplet of the sample and the liquid system, wherein the wave comprises modes encoding characteristics of the interaction between the sample and the liquid system,
   wherein the sample contains the biomolecule, or a composition thereof, for analysis;
ii) determining enthalpies, kinetics and/or timescales of interaction of the sample and the liquid system based on the wave data.
   In yet another aspect, there is provided a method of identifying a candidate biomolecule within a class of biomolecules, or a composition thereof, with comparable enthalpies, kinetics and/or timescales of interaction to a target biomolecule of the same class of biomolecule, or composition thereof, said method comprising the steps of:
(i) obtaining wave data for a sample based on physical measurements of a wave generated, in a liquid system, by providing contact between a droplet of the sample and the liquid system, wherein the wave comprises modes encoding characteristics of the interaction between the sample and the liquid system,
wherein the sample contains a biomolecule candidate of a certain class of biomolecules, or composition thereof, for analysis;
(ii) providing a comparison of the wave data obtained from interaction of the sample with the surface of the liquid system with wave data associated with the target biomolecule, or target composition thereof;
   optionally repeating steps i) to ii) for a different biomolecule candidate of the same class, or composition thereof, until a biomolecule candidate, or composition thereof, is identified having comparable values of at least one form of enthalpy of interaction, indicative of comparable biophysical properties of the candidate biomolecule and the target biomolecule.
   The present invention will now be illustrated by way of the following examples and with reference to the figures.

### General Method for Sample Preparation, Deposition and Wave Data Gathering

1. Obtain a biomolecule, or composition thereof, for testing and prepare a solution, suspension, or emulsion of the biomolecule in a liquid carrier at a w/v concentration of 0.1 mg/ml to 0.5 mg/ml in a sample tube (e.g. a LowBind Eppendorf tube);
2. Prepare a reservoir liquid medium and optionally degas under vacuum;
3. Incorporate the reservoir medium into a reservoir (e.g. a Langmuir trough) into which the sample may be deposited;
4. Deposit a set volume (e.g. 1 to 100 μl) in the form of one or more droplets of the sample into the reservoir using a droplet provider (e.g. a single-channel pipette) from a fixed location above the air-water interface of the reservoir (at a height of 2 to 5 mm above the air-water interface) and at a fixed volumetric flow rate (e.g. 0.1 to 20 μl/s) affording a fixed droplet frequency—i.e. the number of droplets produced per second—(e.g. 0.1 to 1000 droplets/s);
5. Measure the response of the reservoir air-water interface to the first droplet, and any successive droplets, dispensed into the reservoir from the fixed location, using an apparatus incorporating: i) a light beam optics comprising a source of polarised light arranged to illuminate an area of liquid at the surface of the reservoir with a beam having a selected angle of incidence a; ii) a light collector arranged to receive a beam of light after reflection by the area of the thin film, and iii) a detector configured to sense parameters of the light received from the light collector to provide signals to a wave measurement module 17 (for example, an apparatus as shown in FIG. 2 and described in further detail herein).

As will be appreciated, deposition of the sample may be automated and an Opentrons® system (e.g. the OT-1 or OT-2 produced by Opentrons Labworks) may suitably be used for such purposes. The Opentrons® system may accommodate multiple independently prepared samples in distinct sample holders positioned inside the Opentrons System. In particular, the system may be programmed so as to specify:
A. sample location within the Opentrons® system;
B. location from which to dispense sample into a reservoir;
C. height of the droplet provider before dispensing the sample into a reservoir;
D. volumetric flow rate of the sample being dispensed (microlitres per second; and
E. volume of sample to aspirate and dispense (microlitres).

The ability to control the height of the droplet provider and the volumetric flow rate of the sample being dispensed allow for control of droplet size and droplet frequency. During operation, a sample which has been placed inside the Opentrons® system at its specified location is aspirated and then moved to a specified location (x,y), before being moved in a z-direction to a specified z-location. The system pauses and then begins to dispense sample, for example using the Opentrons® 1000 microlitre Single Channel pipette, at the specified volumetric flow rate until the specified volume is fully dispensed.

### Example 1: Glyceride Oil Evaluation

The properties of a number glyceride oils were investigated following the general method described above. Samples of various oils were prepared and tested including: i) Glyceride Oil 1, ii) Glyceride Oil 2, iii) Glyceride Oil 3, iv) Glyceride Oil 4, v) Glyceride Oil 5, and Vi) Glyceride Oil 6. Each oil is a complex mixture of different glyceride oils with saturated, monosaturated and poly-unsaturated fatty acid chains, as well as a minor amount of free fatty acids.

Samples containing the different oils were prepared in the same manner and at the same concentration—1 mg/mL. For each oil investigated, 50 mgs of each oil was weighed into respective 50 mL glass beakers, and deionized and purified water was poured into each beaker to make up the weight of the mixture up to 50 g. Each glass beaker was then placed into a heated sonicator bath (50° C.) and sonicated (operating at 40 kHz, with an ultrasonic power output of 120 W) for 10 minutes with stirring, after which time the mixtures appeared white and opaque, indicating self-assembly of micelles in the water continuous phase, in response to the heat and agitation. 1.5 ml of each mixture was then aliquoted into separate Eppendorf tubes, allowed to equilibrate to room temperature over a 10-minute time-period and placed in the Opentrons® OT-2 system for testing.

The reservoir medium for use in testing in these experiments was the same stock saltwater solution. The reservoirs for use in these experiments were each of dimensions 120 mm×80 mm and filled with the saltwater, with a liquid height alignment of the reservoir being performed optically (using trigger laser intensity, monitored as reservoir medium is extracted from the reservoir until a fixed trigger laser intensity is reached) before the start of each experiment to ensure consistency. The Opentrons® OT2 Workflow, equipped with an Opentrons® 1000 microlitre Single Channel pipette (polypropylene), was used for sample deposition and an apparatus substantially as shown in FIG. 2 and described herein was used for obtaining surface wave data.

The Opentrons® system was set to give a sample deposition volume of 100 μL (as a series of droplets), from a height of less than 1 mm, and to include an isopropanol and water wash between each oil sample testing. During the experiment, the surface pressure of the film at the air-water interface was measured with a Whilhelmy plate, to independently verify the increase in surface pressure as sample deposition occurs, before eventual saturation of the surface with the sample forming a thin film.

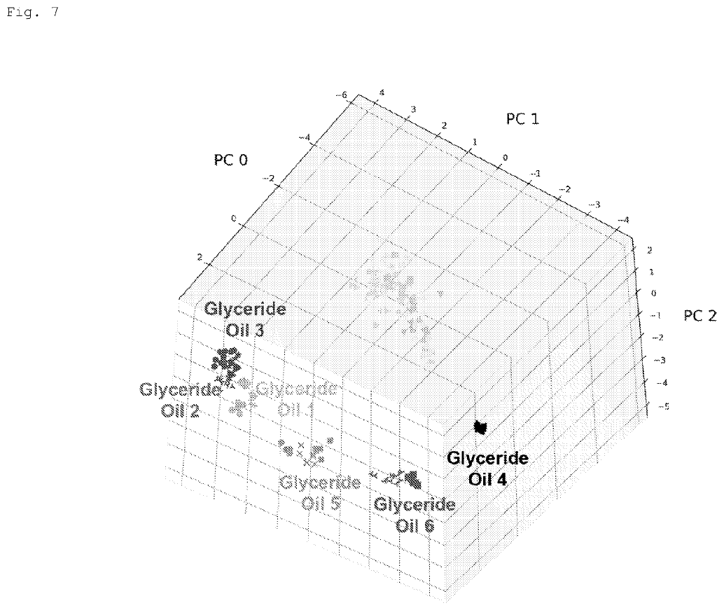

Surface wave data generated from these experiments was subsequently used to generate a biophysical characterisation of each of the oils, incorporating principal component analysis (PCA). The results, which are represented visually in FIG. 7, demonstrate that the present invention allows a unique signature for different biomolecules (in this case glyceride oils), or compositions thereof, to be acquired, representing a combination of biophysical properties of the oil sample. This can be seen in the plot in FIG. 7 which shows distinct clustering for individual oil samples. These data may be used to populate a library of such signatures for comparison purposes, and/or used as means to compare against corresponding data of a target biomolecule to determine whether the biomolecule investigated has comparable biophysical properties.

PCA was found to reveal a direct correlation between the measured signatures for the oil samples of these experiments and the measured surface pressure, as well as correlations between the principal components with pre-determined sensory data for each of the oils investigated (as assessed by a panel of human testers). The correlation between the principal components from the wave data and the pre-determined sensory data indicate that different principle components correlate with different sensory properties, including those selected from skin absorption, skin spread, skin stickiness, skin thickness, in use limpness, in use shine, in use smoothness, in use softness, post wash frizz, post wash shine post wash smoothness, and post wash softness. For example, it was found that (in a Pearson correlation) the zeroth principle component correlates with skin absorption, the first with post-wash smoothness/post-wash softness, and the third with in-use shine.

The correlations in the PCA were validated by correlations between independently determined biophysical and sensory properties of the oils, including correlations between density, viscosity, Iodine value, molecular weight, mono-unsaturated fatty acid content (MUFA), poly-unsaturated fatty acid content (PUFA) and surface tension of the oils. Thus, the present invention enables a biophysical signature to be readily accessed by means of a single test (as opposed to a series of distinct biophysical assessments) from which correlations may be derived with sensory properties and an understanding of whether comparable performance may be exhibited in comparison with a target molecule, for instance, having a particular sensory profile.

### Example 2: Antibody Evaluation

The properties of a number of different monoclonal antibodies (mAbs) were investigated following the general method described above. Samples of different mAbs were prepared and tested including: Abciximab, Bevacizumab, and Adalimumab, as well as 20 anonymised mAb samples obtained from a third-party.

Abciximab, Bevacizumab, and Adalimumab were sourced from Absolute Antibody Ltd (Cleveland, UK) as 1 mg/mL suspensions in PBS, together with 0.02 vol % ProClin® 300 (3% 5-chloro-2-methyl-4-isothiazolin-3-one (CMIT) and 2-methyl-4-isothiazloin-3-one (MIT) in a salt-free proprietary glycol containing an alkyl carboxylate stabilizer, which is widely available). The commercially sourced Abciximab, Bevacizumab, Adalimumab suspensions were removed from the fridge and allowed to equilibrate to room temperature before being opened and diluted to the desired w/v concentration using PBS in LowBind Eppendorf tubes. Final concentrations of finished samples after dilution were 0.5 mg/mL or 0.1 mg/mL. Anonymised mAb samples were provided as 0.1 mg/mL suspensions in PBS by the third-party supplier.

The reservoir medium for use in testing in these experiments was the same stock solution of an anionic surfactant comprising a sulfate group.

The reservoirs for use in these experiments were of dimensions 40 mm×80 mm and filled with the stock aqueous solution, a liquid height alignment of the reservoir was performed optically (using trigger laser intensity, monitored as reservoir medium is extracted from the reservoir until a fixed trigger laser intensity is reached) before the start of each experiment to ensure consistency. The Opentrons® OT2 Workflow, equipped with an Opentrons® 1000 microlitre Single Channel pipette (polypropylene), was used for sample deposition and an apparatus substantially as shown in FIG. 2 and described herein was used for obtaining surface wave data.

The Opentrons® system was set to give a sample deposition volume of 100 μL (as a series of droplets), from a height of less than 1 mm, and to include an isopropanol and water wash between each oil sample testing. During the experiment, the surface pressure of the film at the air-water interface was measured with a Whilhelmy plate, to independently verify the increase in surface pressure as sample deposition occurs, before eventual saturation of the surface with the sample.

Surface wave data generated from these experiments was subsequently used to generate a biophysical characterisation for each of the mAbs, including the three commercially available mAbs and the 20 anonymised mAbs from the third-party supplier. A comparison of the wave data for the three commercially available mAbs was subsequently possible with the wave data of the 20 anonymised samples, allowing an assessment of the comparability between each of those samples and the commercially available mAbs.

An aspect of the disclosure provides an apparatus for biophysical characterisation of a biomolecule of a certain biomolecule class, or a composition thereof, said apparatus comprising:
an analytical instrument configured and arranged to perform physical measurements of a wave in a liquid system, and to obtain, from said measurement, wave data for the sample based on physical measurements of the wave generated, in the liquid system, by providing contact between the liquid system and a droplet of a sample comprising the biomolecule or a composition thereof, wherein the wave comprises modes encoding characteristics of the interaction between the sample and the liquid system, and the apparatus comprises
a data processor configured to provide a biophysical characterisation of the biomolecule or the composition comprising the biomolecule based on the wave data. The analytical instrument may comprise a droplet provider configured to contact the liquid with a droplet of the sample. The analytical instrument may comprise a wave measurement module configured to provide the wave data based on measurements of the wave. The measurements of the wave may be based on interaction of a light beam, such as a laser with the liquid system.

The analytical instrument may comprise a trough holding the liquid system and the liquid system may comprise a thin film. The data processor may be connected to receive the wave data from the analytical instrument. The data processor may also be connected to a data store, such as a digital memory, storing association data. The analytical instrument may be configured to generate wave data comprising a representation of a plurality of said waves generated in the liquid system, each of those waves corresponding to contact of a different one of a plurality of droplets with the liquid system. The data processor may be configured to provide the biophysical characterisation by identifying a plurality of separable contributions to the variance of the wave data. For example, the data processor may be coupled to communicate with data storage storing association data defining a relation between at least one of the separable contributions and a biophysical characteristic. The data processor may be configured to determine data indicating the biophysical characteristic based on the separable contributions and the association data. It is contemplated that the apparatus described in this paragraph is to be configured to perform any one of the methods described or claimed herein.

The present disclosure provides a process for manufacturing a product comprising or made from a biomolecule of a certain biomolecule class, or a composition thereof, the process comprising:
obtaining wave data for a sample based on physical measurements of a wave generated, in a liquid system, by providing contact between a droplet of the sample and the liquid system,
wherein the sample is a liquid comprising a precursor or component of the product, the precursor or component comprises the biomolecule or composition thereof and the wave comprises modes encoding characteristics of the interaction between the sample and the liquid system,
providing a biophysical characterisation of the biomolecule or the composition comprising the biomolecule based on the wave data; and manufacturing the product.

The biophysical characterisation may be provided according to any one of the methods described or claimed herein. The manufacturing may be done according to the biophysical characterisation. The component may comprise a part or all of the product, or an ingredient or other constituent part of the product. The precursor may comprise a substance from which the product is to be made or which is used in the manufacture of the product.

The biophysical characterisation may provide information for use in making the product or for use with the product. For example, the information may be used to verify quality or purity or to adjust a step in the manufacturing process. For example, the product can then be made by making use of said information, such as to control or to verify the quality of said manufacturing process. As another example, the composition and/or the component or precursor may be adjusted based on the information. This may comprise varying a ratio of excipients or constituents of the composition and/or the component or precursor.

Any feature of any one of the examples disclosed herein may be combined with any selected features of any of the other examples described herein. For example, features of methods may be implemented in suitably configured hardware, and the configuration of the specific hardware described herein may be employed in methods implemented using other hardware.

It will be appreciated from the discussion above that the embodiments shown in the Figures are merely exemplary, and include features which may be generalised, removed or replaced as described herein and as set out in the claims. With reference to the drawings in general, it will be appreciated that schematic functional block diagrams are used to indicate functionality of systems and apparatus described herein. It will be appreciated however that the functionality need not be divided in this way, and should not be taken to imply any particular structure of hardware other than that described and claimed below. The function of one or more of the elements shown in the drawings may be further subdivided, and/or distributed throughout apparatus of the disclosure. In some embodiments the function of one or more elements shown in the drawings may be integrated into a single functional unit.

In some examples the functionality of the controllers and data processor described herein may be provided by a general-purpose processor, which may be configured to perform a method according to any one of those described herein. In some examples it may comprise digital logic, such as field programmable gate arrays, FPGA, application specific integrated circuits, ASIC, a digital signal processor, DSP, or by any other appropriate hardware. In some examples, one or more memory elements can store data and/or program instructions used to implement the operations described herein. Embodiments of the disclosure provide tangible, non-transitory storage media comprising program instructions operable to program a processor to perform any one or more of the methods described and/or claimed herein and/or to provide data processing apparatus as described and/or claimed herein. The controllers and data processors may comprise an analogue control circuit which provides at least a part of this control functionality. An embodiment provides an analogue control circuit configured to perform any one or more of the methods described herein.

The above embodiments are to be understood as illustrative examples. Further embodiments are envisaged. It is to be understood that any feature described in relation to any one embodiment may be used alone, or in combination with other features described, and may also be used in combination with one or more features of any other of the embodiments, or any combination of any other of the embodiments. Furthermore, equivalents and modifications not described above may also be employed without departing from the scope of the invention, which is defined in the accompanying claims.

1. A method of biophysical characterisation of a biomolecule of a certain biomolecule class, or a composition thereof, said method comprising the steps of:
i) obtaining wave data for a sample based on physical measurements of a wave generated, in a liquid system, by providing contact between a droplet of the sample and the liquid system, wherein the wave comprises modes encoding characteristics of the interaction between the sample and the liquid system,
wherein the sample contains the biomolecule, or a composition thereof, for analysis;
ii) providing a biophysical characterisation of the biomolecule or the composition comprising the biomolecule based on the wave data.

2. A method of identifying a candidate biomolecule within a class of biomolecules, or a composition thereof, with comparable biophysical properties to a target biomolecule of the same class of biomolecule, or composition thereof, said method comprising the steps of:
(i) obtaining wave data for a sample based on physical measurements of a wave generated, in a liquid system, by providing contact between a droplet of the sample and the liquid system, wherein the wave comprises modes encoding characteristics of the interaction between the sample and the liquid system,
wherein the sample contains a biomolecule candidate of a certain class of biomolecules, or composition thereof, for analysis;
(ii) providing a comparison of the wave data obtained from interaction of the sample with the surface of the liquid system with wave data associated with the target biomolecule, or target composition thereof;
optionally repeating steps i) to ii) for a different biomolecule candidate of the same class, or composition thereof, until a biomolecule candidate, or composition thereof, is identified having comparable values of at least one biophysical characteristic, indicative of comparable biophysical properties of the candidate biomolecule and the target biomolecule.

3. The method of claim 2, wherein the comparison is based on a biophysical characterisation of the biomolecule or the composition comprising the biomolecule based on the wave data and a corresponding characterisation of the target biomolecule, or target composition thereof.

4. The method of claim 1 or 3 wherein the wave data comprises a representation of a plurality of said waves generated in the liquid system, each of those waves corresponding to contact of a different one of said droplets with the liquid system.

5. The method of claim 1, 3 or 4 wherein providing a biophysical characterisation comprises identifying a plurality of separable contributions to the variance of the wave data.

6. The method of claim 5 comprising:
obtaining association data defining a relation between at least one of the separable contributions and a biophysical characteristic, and
determining data indicating the biophysical characteristic, based on the separable contributions and the association data,
wherein the biophysical characterisation of the biomolecule or a composition comprising the biomolecule provided at step (iii) comprises the data indicating the biophysical characteristic.

7. The method of claim 5 or 6 comprising applying a dimensionality reduction method to the wave data to identify the separable contributions.

8. The method of claim 7 wherein the dimensionality reduction method comprises a blind signal separation, BSS, method.

9. The method of claim 8 wherein the BSS method comprises at least one method selected from the list comprising:
principal component analysis;
singular value decomposition;
independent component analysis; and
non-negative matrix factorization.

10. The method of claim 9 wherein the dimensionality reduction method comprises a multi-resolution analysis method.

11. The method of claim 10 wherein the multi-resolution analysis method comprises at least one method selected from the list comprising:
a time-frequency analysis, such as analysis based on a short-time fourier transform a wavelet analysis, such as analysis based on a short-time fourier transform

12. The method of any preceding claim wherein the class of biomolecules is selected from polymeric and/or macromolecular classes of biomolecules.

13. The method of claim 12 wherein the class of biomolecules is selected from polypeptides, lipids, polysaccharides, and nucleic acids.

14. The method of claim 13 wherein the class of biomolecules is selected from proteins.

15. The method of claim 14, wherein the class of biomolecules is selected from antibodies, contractile proteins, enzymes, hormonal proteins, structural proteins, storage proteins, and transport proteins.

16. A method according to claim 14 wherein the biomolecule, biomolecule candidate, or composition thereof is i) a plant protein selected from algae protein, black bean protein, canola wheat protein, chickpea protein, fava protein, lentil protein, lupin bean protein, mung bean protein, oat protein, pea protein, potato protein, rice protein, soy protein, sunflower seed protein, wheat protein, white bean protein, and protein isolates or concentrates thereof; or ii) an animal protein selected from porcine protein, bovine protein, ovine protein, equine protein, avian protein and protein isolates or concentrates thereof.

17. The method of cany of claims 1 to 11 wherein the class of biomolecules is selected from non-polymeric and/or non-macromolecular classes of biomolecules.

18. The method of claim 17 wherein the class of biomolecules is selected from vitamins, phytochemicals, food flavourings, amino acids, peptides, mono-, di- and oligo-saccharides, free fatty acids, mono-, di-, and triglycerides; sterols, steroids, nucleotides and nucleosides.

19. The method of claim 18, wherein the class of biomolecules is an edible glyceride oil selected from vegetable oils, marine oils, and animal oils.

20. The method of claim 19 wherein the biomolecule, biomolecule candidate, or composition thereof, is a vegetable oil selected from açai oil, almond oil, beech oil, cashew oil, coconut oil, colza oil, corn oil, cottonseed oil, grapefruit seed oil, grape seed oil, hazelnut oil, hemp oil, lemon oil, macadamia oil, mustard oil, olive oil, orange oil, peanut oil, pecan oil, pine nut oil, pistachio oil, poppyseed oil, rapeseed oil, rice bran oil, safflower oil, sesame oil, soybean oil, sunflower oil, walnut oil and wheat germ oil.

21. The method of any preceding claim wherein the biophysical characteristic comprises a sensory property of the biomolecule, or a composition thereof.

22. The method of claim 21 wherein the biomolecule is an edible glyceride oil selected from vegetable oils/fats, marine oils/fats, and animal oils/fats, wherein the sensory property indicates a property associated with application of the biomolecule, or a composition thereof, to the human or animal body.

23. The method of claim 22 wherein the sensory property indicates least one of: skin absorption, skin spread, skin stickiness, skin thickness, in use limpness, in use shine, in use smoothness, in use softness, post wash frizz, post wash shine post wash smoothness, and post wash softness.

24. The method of claim 21 wherein the biomolecule is selected from the proteins class.

25. The method of claim 24 wherein the sensory property indicates a property exhibited by the biomolecule or a composition comprising the biomolecule in use in a food product.

26. The method of any of claims 1 to 20, wherein the biomolecule, or composition thereof, is a therapeutic candidate.

27. The method of claim 26 wherein the biomolecule is a protein, such as an antibody or hormonal protein.

28. The method of claim 26 or 27 wherein the biophysical characteristic comprises a metric of developability, such as manufacturability or safety profile.

29. The method of claim 28 wherein the metric of developability is based on a plurality of biophysical characteristics, said plurality of biophysical characteristics each being derived from one or more separable contributions of the wave data.

30. The method of any preceding claim wherein the biophysical characteristic of the biomolecule, or composition thereof, is selected from viscosity, stability in a particular environment, hydrophobicity, binding affinity to a particular target, solubility in a particular solvent, surface charge, and specific gravity.

31. The method of any preceding claim further comprising performing the physical measurements of the wave.

32. The method of any preceding claim further comprising providing the contact between the droplet of the sample and the liquid system to generate the wave.

33. The method of any preceding claim further comprising preparing the sample for analysis.

34. The method of any preceding claim wherein the wave comprises a surface wave.

35. The method of claim 34 wherein the modes encoding characteristics of the interaction between the sample and the liquid system comprises a Lucassen wave mode and the physical measurement comprises a sensitivity to said Lucassen wave mode.

36. A process for manufacturing a product comprising or made from a biomolecule of a certain biomolecule class, or a composition thereof, the process comprising:
obtaining wave data for a sample based on physical measurements of a wave generated, in a liquid system, by providing contact between a droplet of the sample and the liquid system,
wherein the sample is a liquid comprising a precursor or component of the product, the precursor or component comprises the biomolecule or composition thereof and the wave comprises modes encoding characteristics of the interaction between the sample and the liquid system, providing a biophysical characterisation of the biomolecule or the composition comprising the biomolecule based on the wave data; and
manufacturing the product.

37. The process of claim 36 wherein the biophysical characterisation is provided by the method of any of claims 1 to 35.
